(19) United States
(12) Patent Application Publication
(10) Pub. No.: US 2026/0348422 A1
(43) Pub. Date: Nov. 5, 2026

(54) WIRE FIXING MECHANISM AND ENDOSCOPE USING THE SAME

(71) Applicant: Medimaging Integrated Solution, Inc., Hsinchu (TW)

(72) Inventors: YI-MING TAI, Hsinchu (TW); YUEH-YING FAN, Hsinchu (TW); YU-TSENG LEE, Hsinchu (TW)

(21) Appl. No.: 19/340,430

(22) Filed: Sep. 25, 2025

(30) Foreign Application Priority Data
May 2, 2025 (TW) 114116574

(51) Int. Cl. F16C 1/10; B25J 9/00

(52) U.S. Cl. CPC F16C 1/102; B25J 9/0009

(57) ABSTRACT

A wire fixing mechanism and an endoscope using the same are provided. The wire fixing mechanism includes a base, fixing members, securing members, and a first glue. The fixing members are formed on both sides of the base and are respectively provided with coaxial first and second recessions, with the first recession located above the second recession. The sidewall of the first recession is provided with a first opening and a second opening for wire insertion and passage, respectively. Each of the securing members includes a shaft and a head, which are fabricated into a one-piece component, wherein the shaft is inserted into the second recession, and the head is inserted into the first recession to press against and fix the wire. The first glue is disposed between the head and the first recession to enhance the fixing effect of the wire.

### BACKGROUND OF THE INVENTION

### 1. Field of the Invention

The present invention relates to an endoscopic technology, particularly to a wire fixing mechanism used to fix internal wire of an endoscope and an endoscope using the same.

### 2. Description of the Prior Art

In the conventional technology, wire is usually used to control and operate the bendable section of an endoscope. After being installed, the wire should be tensed and fixed to a member of the control end to guarantee structural stability and operational dexterity. One of the methods of fixing the wire is to pass the wire through the member of the control rod, loop the wire tightly and then use a metal ring to grip the wire fixedly. In the abovementioned method, not only additional space needs to be preserved for looping the wire, but also abrasion and fatigue is likely to occur in the bent region of the wire; thus, the durability of the wire is reduced. Another method is to pass the wire through the direction guide structure or let the wire go around the path to fix the wire and set the tension of the wire. In the abovementioned method, not only the complexity of designing the endoscope is increased, but also over bending and abrasion is likely to wear and fatigue the wire. Besides, in the processes of assembling the wire and calibrating the tension of the wire, the looping-fixing steps are complicated and hard to achieve consistent precision. Further, the conventional technology also impairs future maintenance and repairment.

Accordingly, the present invention proposes a wire fixing mechanism and an endoscope using the same to overcome the conventional problems.

### SUMMARY OF THE INVENTION

One objective of the present invention is to provide a wire fixing mechanism and an endoscope using the same, which neither have the complicated structure of the conventional technology nor use additional diversion structure (such as guiding walls, guiding pins, or rollers), whereby to downsize the overall module and simplify the fabrication process, and whereby to prevent the fixed position of the wire from abrasion and stress concentration, which is induced by looping or overbending, wherefore the service life of the wire is increased.

In order to achieve the abovementioned objective, the present invention provides a wire fixing mechanism, which comprises a base, fixing members, securing members, and a first glue. The fixing members are disposed at two sides of the base. Each of the fixing members includes a first recession and a second recession, which are coaxially arranged, and the first recession is located above the second recession. The diameter of the first recession is greater than the diameter of the second recession. A first opening and a second opening are formed on the sidewall of the first recession. One end of a wire is inserted through the first opening. The same end of the wire is passed through and extended out from the second opening. The securing member is disposed in the fixing member. The securing member includes a shaft and a head, which are integrally fabricated into a one-piece component. The head is connected with one end of the shaft. The shaft is inserted into the second recession, and the head is inserted into the first recession. While the securing member is inserted into the fixing member, the head would be pressed against the wire to fix the wire. The first glue is filled into the gap between the head and the first recession to secure the wire furthermore.

In one embodiment of the present invention, the wire fixing mechanism further comprises a gripping element. The gripping element is disposed at the second opening, pressing against the second opening to grip the wire extended out. The fixing member is extended outward from the second opening to form an accommodation recess. The gripping element is arranged inside the accommodation recess. The edges of the gripping element approach the sidewall of the accommodation recess closely. The surface of the gripping element and the bottom of the accommodation recess jointly form a griping space. The wire is gripped inside the gripping space.

In one embodiment of the present invention, the fixing member is extended outward from the second opening to form an accommodation recess. One sidewall of the accommodation recess has a plurality of open slots. The open slots are equidistantly and parallel arranged to form a comb-like structure. The open slots are extended along the depth direction until they reach the bottom of the accommodation recess, allowing the wire to wind around. The open slots are arranged vertically to the distribution direction or arranged obliquely to the distribution direction at a given tilt angle.

In one embodiment of the present invention, the fixing member is extended outward from the second opening to form an accommodation recess. Two sidewalls of the accommodation recess respectively have a plurality of open slots. The open slots of each sidewall are arranged intermittently to form a comb-like structure, allowing the wire to wind around.

In one embodiment of the present invention, the fixing member is extended outward from the second opening to form an accommodation recess. The bottom of the accommodation recess has a plurality of via-holes. The via-holes are intermittently arranged, allowing the wire to wind around.

In one embodiment of the present invention, the spacing of the open slots or via-holes is 1-1.5 times the diameter of the wire.

In one embodiment of the present invention, the spacing of the open slots or via-holes is smaller than the minimum turning diameter of the looping region of the wire.

In one embodiment of the present invention, the wire fixing mechanism further comprises a second glue. The second glue is filled into the accommodation recess to cover the wire.

In order to achieve the abovementioned objective, the present invention further provides an endoscope, which comprises a housing, the abovementioned wire fixing mechanism, and a wire. The wire fixing mechanism is disposed inside the housing. The wire is also disposed inside the housing. Two ends of the wire are respectively fixed to the wire fixing mechanism, wherein two ends of the wire are respectively passed through two fixing members at two sides of the base, and securing members are respectively inserted into the fixing members to press against and fix the wire.

In summary, the present invention proposes a wire fixing mechanism and an endoscope using the same, which adopt a straight line or straight line-like fixing path design and are exempted from the diversion-guide structure, such as guide walls, guide pins or rollers, whereby to simplify overall structure, reduce fabrication cost, and favor lightweight and miniaturization. Because the fixed position of the wire is neither looped nor diverted abruptly, the stress concentration and abrasion loss induced by bending is reduced. Therefore, the risk of loosening and damage is decreased, and the safety of usage is increased. The present invention provides an easy and convenient fixing method, exempted from the tension adjustment process required by the conventional looping type fixing structure. Therefore, the present invention can obviously raise the assemblage efficiency and product uniformity.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein

FIG. 1 is a perspective view schematically showing a first embodiment of the wire fixing mechanism according to the present invention;

FIG. 2 is a diagram schematically showing a first embodiment of an endoscope according to the present invention;

FIG. 3 is a diagram schematically showing a second embodiment of the wire fixing mechanism according to the present invention;

FIG. 4 is a perspective view schematically showing a third embodiment of the wire fixing mechanism according to the present invention;

FIG. 5 is a diagram schematically showing a local region of the third embodiment of the wire fixing mechanism according to the present invention;

FIG. 6 is a diagram schematically showing an assembling process of the third embodiment of the wire fixing mechanism according to the present invention;

FIG. 7 is a diagram schematically showing a local region of a fourth embodiment of the wire fixing mechanism according to the present invention;

FIG. 8 is a sectional view taken along Line A-A in FIG. 7;

FIG. 9 is a diagram schematically showing another embodiment of the open slots according to the present invention;

FIG. 10 is a diagram schematically showing a local region of a fifth embodiment of the wire fixing mechanism according to the present invention;

FIG. 11 is a diagram schematically showing a local region of a sixth embodiment of the wire fixing mechanism according to the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The embodiments of the present invention will be further demonstrated in details hereinafter in cooperation with the corresponding drawings. In the drawings and the specification, the same numerals represent the same or the like elements as much as possible. For simplicity and convenient labelling, the shapes and thicknesses of the elements may be exaggerated in the drawings. It is easily understood: the elements belonging to the conventional technologies and well known by the persons skilled in the art may be not particularly depicted in the drawings or described in the specification. Various modifications and variations made by the persons skilled in the art according to the contents of the present invention are to be included by the scope of the present invention.

Below is introduced a first embodiment of a wire fixing mechanism according to the present invention.

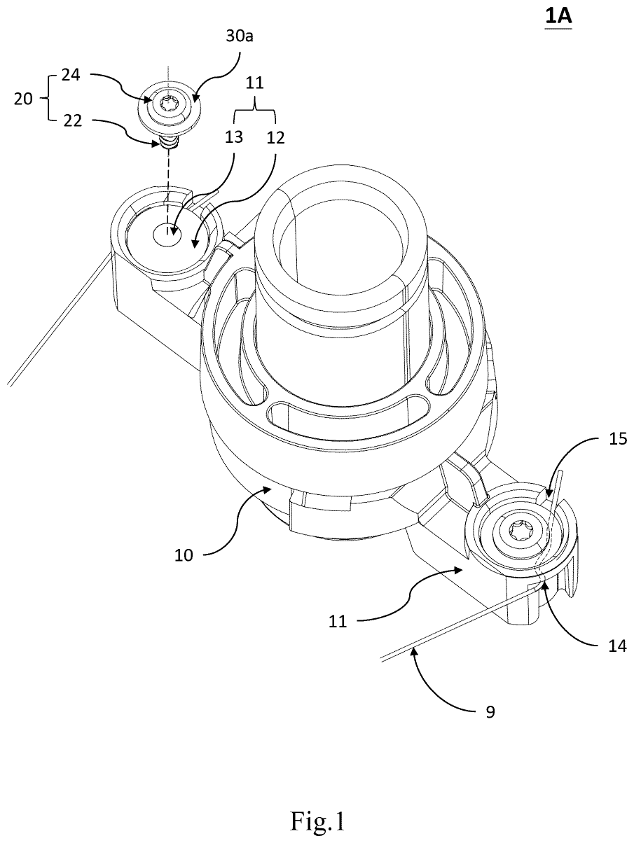

Refer to FIG. 1. The wire fixing mechanism 1A comprises a base 10, fixing members 11, securing members 20, and a first glue 30a. The fixing members 11 are disposed at two sides of the base 10. The fixing member 11 includes a first recession 12 and a second recession 13, which are coaxially arranged, and the first recession 12 is located above the second recession 13. The diameter of the first recession 12 is greater than the diameter of the second recession 13. A first opening 14 and a second opening 15 are formed on the sidewall of the first recession 12. One end of a wire 9 is inserted through the first opening 14. The same end of the wire 9 is passed through and extended out from the second opening 15. In the embodiment shown in FIG. 1, the second opening 15 is in form of a vertical notch. However, the second opening 15 may be but is not limited to be in form of a via-hole, a channel, or a horizontal notch in practical application. The securing member 20 is disposed in the fixing member 11. The securing member 20 includes a shaft 22 and a head 24, which are integrally fabricated into a one-piece component. The head 24 is connected with one end of the shaft 22. The shaft 22 is inserted into the second recession 13, and the head 24 is inserted into the first recession 12. In the embodiment shown in FIG. 1, a screw is used to exemplify the securing member 20. However, the securing member 20 may be but is not limited to be a bolt, a rivet, or a nail in other embodiments. In the case that the securing member 20 is inserted into the fixing member 11, the head 24 would be pressed against the wire 9 to fix the wire 9. The first glue 30a is filled into the gap between the head 24 and the first recession 12 to secure the wire 9 furthermore.

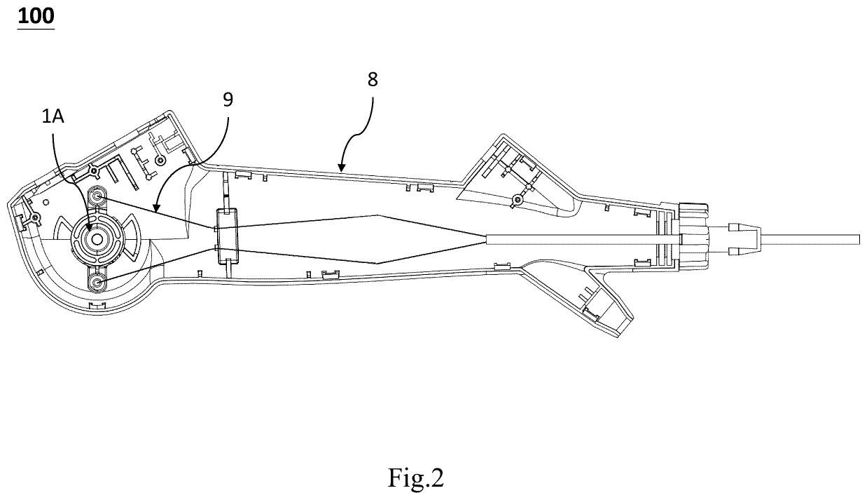

Refer to FIG. 2. The present invention provides an endoscope 100. In order to demonstrate the structural characteristics clearly, FIG. 2 is a schematic diagram mainly showing the control handle where a portion of housing is dismounted. The endoscope 100 comprises a housing 8, the wire fixing mechanism 1A shown in FIG. 1, and the wire 9. The wire fixing mechanism 1A is disposed inside the housing 8. Two ends of the wire 9 are fixed to the wire fixing mechanism 1A, wherein two ends of the wire 9 are respectively passed through the fixing members at two sides of the base of the wire fixing mechanism 1A. The securing member is inserted into the fixing member to press against and fix the wire 9.

It is preferred: the wire fixing mechanism 1A is disposed inside the control handle. In the embodiment, the base is a wheel. The wheel is normally used to control the direction of the far-end camera of the endoscope. While the wheel is rolling, it will pull the wire 9 to vary the direction of the far-end camera.

The present invention simplifies the complicated structure of the traditional wire fixing device. Thereby, the present invention can reduce the cost, raise the fabrication efficiency. Further, the present invention can prevent the wire from being bent at the fixed position, reduce the stress of the wire, and decrease the abrasion loss of the wire. Therefore, the present invention can promote the safety and reliability of operation.

Below are introduced various embodiments of the wire fixing mechanism of the present invention. It should be understood by the persons having ordinary knowledge of the art: the wire fixing mechanisms of various embodiments of the present invention are all applicable to the endoscope 100. Thus, the detailed structure of the endoscope will not repeat below.

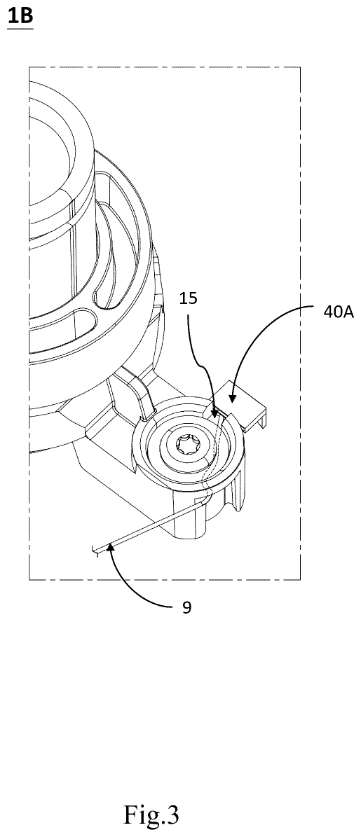

FIG. 3 is a diagram schematically showing a local region of a wire fixing mechanism according to a second embodiment of the present invention. The elements of the second embodiment are the same as those of the first embodiment except the second embodiment has a gripping element 40A. Therefore, the same elements in FIG. 3 are designated with the same symbols as in FIG. 1 and will not repeat herein. Refer to FIG. 3. The wire fixing mechanism 1B further comprises a gripping element 40A. The gripping element 40A is disposed at the second opening 15, pressing against the second opening 15 to grip the wire 9 extended out. The gripping element 40A may be but is not limited to be made of stainless steel, copper, or another metal.

The wire fixing mechanism 1B can fix the tensioned wire 9 without diverting the wire 9, whereby the diversion-induced loss is reduced. The wire fixing mechanism 1B adopt a two-stage fixing method, which can effectively promote the stability and reliability of the wire. Firstly, the wire 9 is guided to the fixing member 11; the securing member 20 fixes the wire 9, and the first glue further provides an auxiliary fixing function, whereby the first fixing stage is completed. Next, the extended wire 9 is inserted into the gripping element 40A, and the gripping element 40A presses against the base 10 to grip the wire 9 tightly, then the first glue 30a is filled into the securing member 20 or the base 10 to support the gripping element 40A, the securing member 20 and the base 10, whereby the second fixing stage is completed. Thus, the engagement of the wire 9 and the wire fixing mechanism 1B becomes tight and secure. The design of two-stage fixing method provided by the present invention not only can promote the operational flexibility but also can adjust the fixing strength according to different applied forces. The two-stage fixing method of the present invention is particularly suitable to apply to the medical endoscopes, etc., which require accurate positioning and experience hauling force.

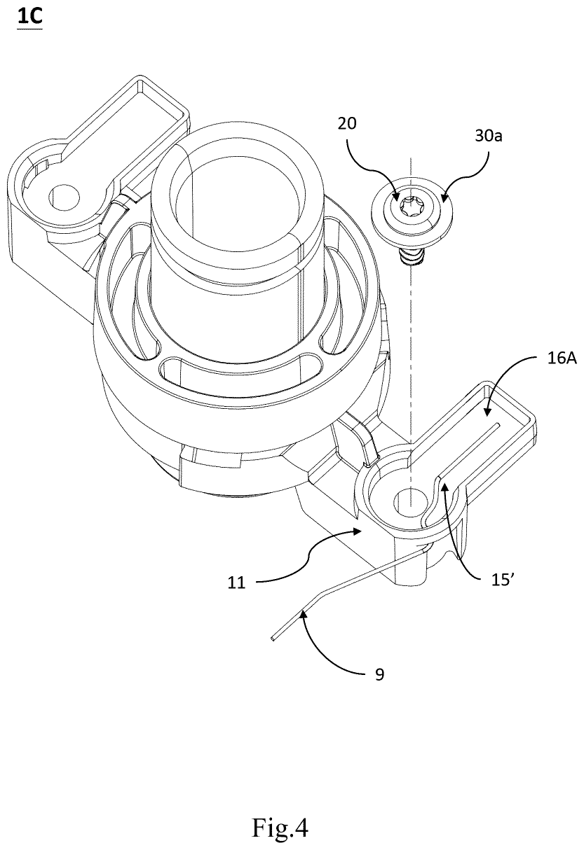

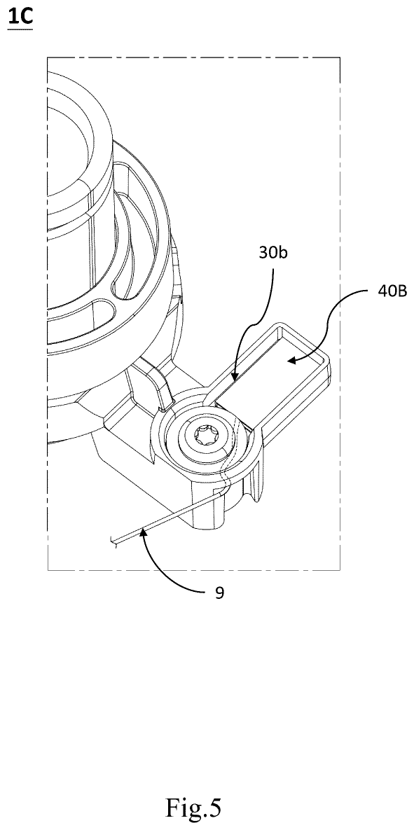

FIG. 4 and FIG. 5 schematically show a third embodiment of the present invention. FIG. 4 is a diagram schematically showing the wire fixing mechanism, wherein a gripping element 40B has not yet been installed in the wire fixing mechanism. FIG. 5 is a diagram schematically showing a local region of the wire fixing mechanism, wherein the gripping element 40B has been installed in the wire fixing mechanism. The wire fixing mechanism 1C of the third embodiment is essentially the same as the wire fixing mechanism 1A of the first embodiment except the wire fixing mechanism 1C further comprises second openings 15′, accommodation recesses 16A, gripping elements 40B, and a second glue 30b. The same elements in the third embodiment are designated with the same symbols as in the first embodiment and will not repeat herein. Refer to FIG. 4. In the third embodiment, the second opening 15′ is exemplified by a passage. The fixing member 11 is extended outward to form the accommodation recess 16A. The accommodation recess 16A interconnects with the first recession 12. The gripping element 40B is arranged inside the accommodation recess 16A. The edges of the gripping element 40B approach the sidewalls of the accommodation recess 16A closely. The surface of the gripping element 40B and the bottom of the accommodation recess 16A jointly form a griping space. The wire 9, which passes through second opening 15′, is extended into the gripping space and gripped by the gripping element 40B. The second glue 30b is filled into the accommodation recess 16A to cover the wire 9 and enhance the fixing effect. The accommodation recess 16A and the fixing member 11 may be integrally fabricated into a one-piece component. The accommodation recess 16A is a platform-like structure; the space surrounded by the sidewalls and the bottom of the accommodation recess 16A may receive the wire 9, the gripping element 40B, and the second glue 30b.

In this embodiment, the gripping element 40B is a U-shaped structure. In other embodiments, the gripping element 40B may be but is not limited to be in form of a metal spring plate, a cylindrical structure, or a U-shaped fastener.

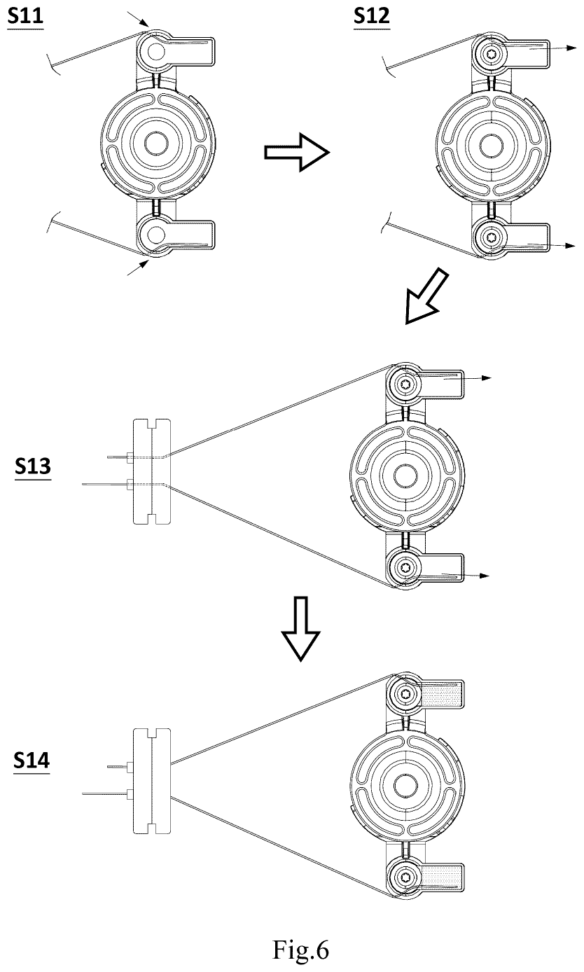

FIG. 6 is a diagram schematically showing the assemblage steps of the wire fixing mechanism of the third embodiment. Refer to FIG. 6. The assemblage steps include

Step S11: insert the wire into the first recession of the base (wheel);

Step S12: pre-lock the securing member (screw), but don't lock it tightly;

Step S13: pull the wire to the required tension; apply the glue liquid to the space between the screw and the wire, and lock the screw tightly;

Step S14: use a gripping element (such as a metal ring) to grip and fix the terminal of the wire and let the terminal be pressed against by the screw; then use a glue liquid to stick the terminal to the housing or the edge of the fixing member (the sidewall).

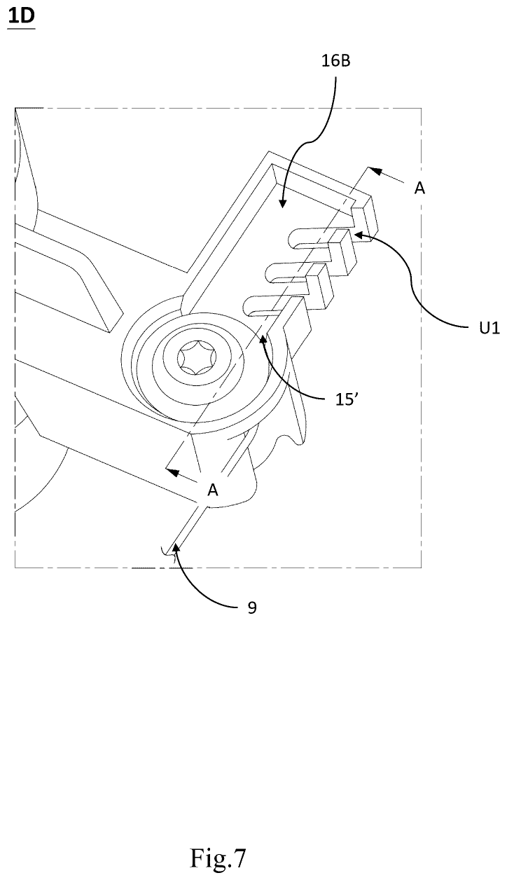

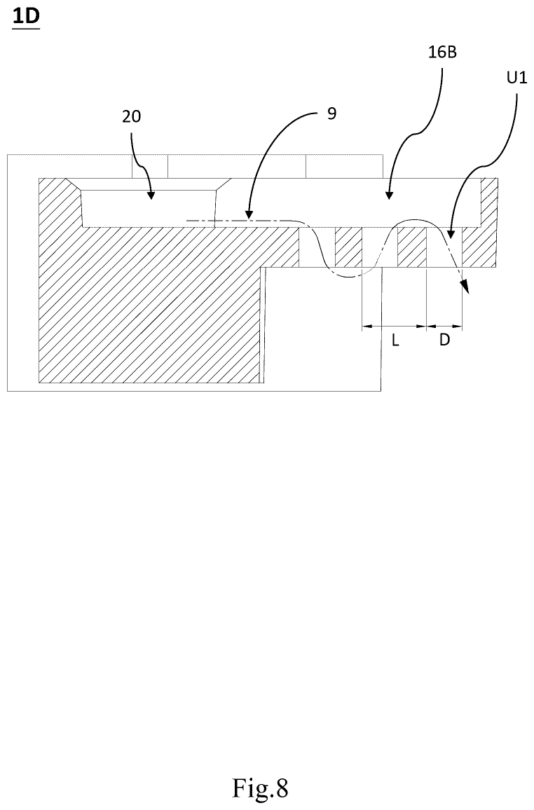

FIG. 7 and FIG. 8 are diagrams schematically showing a wire fixing mechanism according to a fourth embodiment of the present invention. The elements of the wire fixing mechanism 1D of the fourth embodiment are the same as those of the third embodiment except the wire fixing mechanism 1D of the fourth embodiment has an accommodation recess 16B. The same elements will not repeat herein. Refer to FIG. 7 and FIG. 8. None gripping element is used in the fourth embodiment. One sidewall of the accommodation recess 16B has a plurality of open slots U1. The slots are arranged intermittently to form a comb-like structure. The open slots U1 are extended along the depth direction until they reach the bottom of the accommodation recess 16B to form narrow openings, which allow the wire to be wound around and fixed to. The comb-like structure can make the wire be arranged neatly and prevent the wire from being displaced. While used in an endoscope, the wire fixing mechanism 1D can effectively decrease the noise induced by the overlapped wire and reduce the friction force experienced in the diversion of the wire.

The wire 9 may penetrate out through the second opening 15′ to complete the first-stage fixing operation and extend into the accommodation recess 16B. Then, the wire 9 is wound around the plurality of open slots U1, wherein the wire 9 may be interlaced horizontally or vertically. Suppose the spacing between two open slots U1 is L and the minimum turning radius of the wire 9 is Rmin. Thus, the spacing between two open slots U1 should be smaller 2Rmin, i.e. L<2Rₘᵢₙ. Suppose the width of the open slot U1 is D and the diameter of the wire 9 is d. Thus, the width of the open slot U1 should meet the formula: d≤D≤1.5d.

Via the structural design of the open slots U1, the tension force of the wire 9 may generate a friction force between the wire 9 and the wheel of the base 10. After winding, the wire 9 may be fixed to the base 10 with the second glue, whereby a pull-resistant force is generated to provide the second-stage fixing effect.

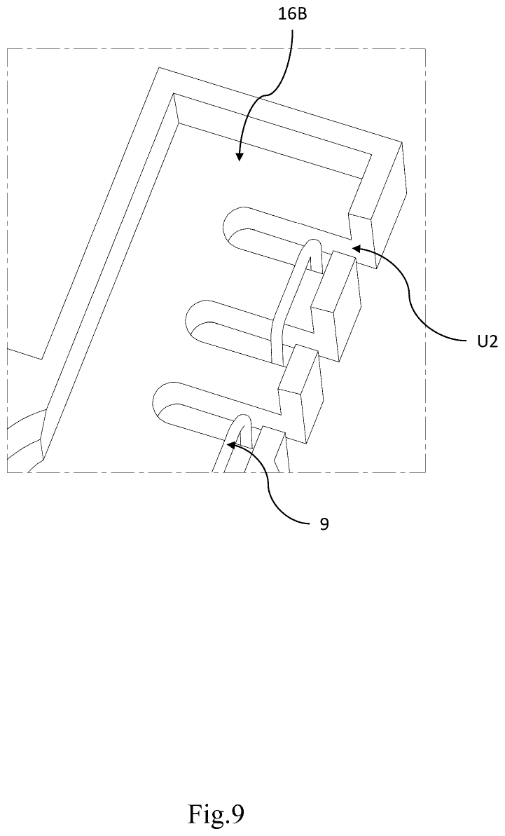

FIG. 9 schematically shows another embodiment of the open slots. The slots U2 of the embodiment in FIG. 9 is different from the slots U1 of the embodiment in FIG. 7 in the angles of the slots. Refer to FIG. 7 and FIG. 9 simultaneously. The open slots U1 are slanted slots distributed parallel, wherein the open slots U1 are slanted by a given angle to the distribution direction to form a slanted open slot structure. The open slots U2 are vertical slots distributed parallel to form a comb-like structure, wherein the open slots U2 are parallel and equidistant, and wherein the openings of the open slots are formed vertically to the distribution direction.

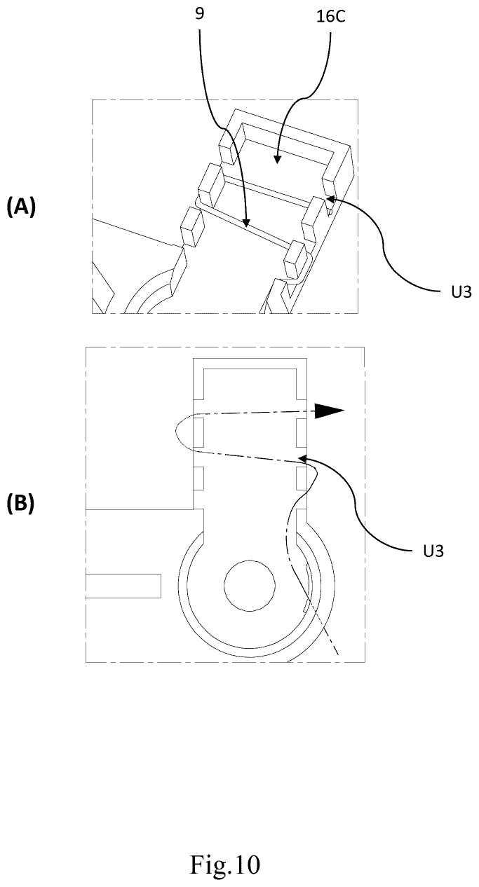

FIG. 10 schematically shows a fifth embodiment of the wire fixing mechanism of the present invention. The elements of the wire fixing mechanism of the fifth embodiment are the same as those of the fourth embodiment except the wire fixing mechanism of the fifth embodiment has an accommodation recess 16C. The same elements will not repeat herein. Refer to FIG. 10. Two sidewalls of the accommodation recess 16C respectively have a plurality of open slots U3. The open slots U3 of each sidewall are arranged intermittently to form a comb-like structure. The wire 9 may be wound around the open slots U3 respectively on two sidewalls and interwoven horizontally between two sidewalls.

As shown in FIG. 10 (A) and FIG. 10 (B), two sidewalls of the accommodation recess 16C respectively have a plurality of protrusions arranged intermittently to form staggered teeth-like structures, whereby to increase the contact area between the wire 9 and the accommodation recess 16C. Thus, the reliability of fixing the wire is further enhanced.

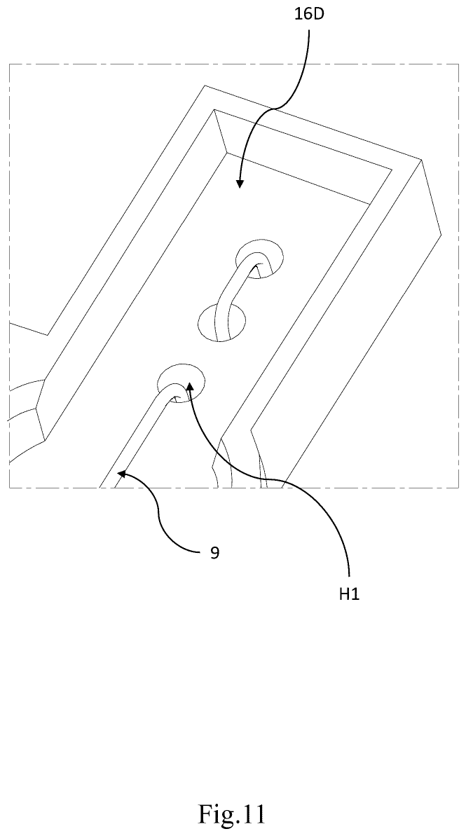

FIG. 11 schematically shows a sixth embodiment of the wire fixing mechanism of the present invention. The elements of the wire fixing mechanism of the sixth embodiment are the same as those of the fourth embodiment except the wire fixing mechanism of the sixth embodiment has an accommodation recess 16D. Refer to FIG. 11. The bottom of the accommodation recess 16D has a plurality of via-holes H1. The plurality of via-holes H1 is arranged intermittently, allowing the wire 9 to wind. It is preferred: the diameter of the via-hole H1 is 1-1.5 times the diameter of the wire 9; the spacing of the via-holes H1 is smaller than the minimum turning diameter of bending the wire 9, whereby to prevent the wire 9 from creasing or fracturing during bending.

In conclusion, the wire fixing mechanism and the endoscope of the present invention adopt a fixing method, which does not use a looping structure or a diversion-guide structure but directly passes the tensed wire through a predetermined path, wherein a securing structure is further used to fix the wire. Compared with the conventional technology, the present invention has the following advantages and efficacies:
(1) Simplifying the structure: the fixing path of the wire is a straight line or nearly a straight line, exempted from any additional diversion-guide structure, such as guide walls, guide pins or rollers, whereby to reduce fabrication cost and favor lightweight and miniaturization;
(2) Decreasing the extent of wire bending and wire abrasion: the present invention avoids looping or diverting the wire at the fixed position to effectively reduce stress concentration and abrasion loss, whereby to prevent from the wire loosening and medical accident during operating the endoscope;
(3) Increasing assembling efficiency and product reliability: it is unnecessary to adjust the winding tension of the wire, and the fixing method is very simple, whereby the assembling efficiency and product uniformity is increased.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

1. A wire fixing mechanism, comprising
a base;
fixing members, respectively disposed at two sides of the base, each including a first recession and a second recession, which are coaxially arranged, wherein the first recession is located above the second recession; a diameter of the first recession is greater than a diameter of the second recession; a first opening and a second opening are formed on a sidewall of the first recession; one end of a wire is inserted through the first opening; the same end of the wire is passed through and extended out from the second opening;
securing members, respectively disposed in the fixing members, each including a shaft and a head, which are integrally fabricated into a one-piece component, wherein the head is connected with one end of the shaft; the shaft is inserted into the second recession; the head is inserted into the first recession; while the securing member is inserted into the fixing member, the head is pressed against the wire to fix the wire; and
a first glue, filled into a gap between the head and the first recession to secure the wire.

2. The wire fixing mechanism according to claim 1, further comprising a gripping element, which is disposed outside the second opening and pressed against the second opening to grip the wire extended out from the second opening.

3. The wire fixing mechanism according to claim 2, wherein the fixing member is extended outward from the second opening to form an accommodation recess; the gripping element is arranged inside the accommodation recess; edges of the gripping element approach sidewalls of the accommodation recess closely; a surface of the gripping element and a bottom of the accommodation recess jointly form a griping space; the wire is gripped inside the gripping space.

4. The wire fixing mechanism according to claim 1, wherein the fixing member is extended outward from the second opening to form an accommodation recess; one sidewall of the accommodation recess has a plurality of open slots; the plurality of open slots has a plurality of slot openings; the slot openings are arranged equidistantly and parallel to form a comb-like structure; the open slots are extended along a depth direction of the accommodation recess toward a bottom of the accommodation recess.

5. The wire fixing mechanism according to claim 4, wherein the open slots are arranged vertically with respect to a distribution direction of the open slots or arranged obliquely by a given tilt angle with respect to the distribution direction of the open slots.

6. The wire fixing mechanism according to claim 1, wherein the fixing member is extended outward from the second opening to form an accommodation recess; each of two sidewalls of the accommodation recess has a plurality of open slots; the open slots of each sidewall are arranged intermittently to form a comb-like structure allowing the wire to wind around.

7. The wire fixing mechanism according to claim 1, wherein the fixing member is extended outward from the second opening to form an accommodation recess; a bottom of the accommodation recess has a plurality of via-holes; the via-holes are arranged intermittently and allows the wire to wind around.

8. The wire fixing mechanism according to claim 4, wherein a width of the open slot is 1-1.5 times a diameter of the wire.

9. The wire fixing mechanism according to claim 4, wherein a spacing of the open slots is smaller than a minimum turning diameter of the wire.

10. The wire fixing mechanism according to claim 7, wherein a diameter of the via-hole is 1-1.5 times a diameter of the wire.

11. The wire fixing mechanism according to claim 7, wherein a spacing of the via-holes is smaller than a minimum turning diameter of the wire.

12. The wire fixing mechanism according to claim 4, wherein a second glue is filled into the accommodation recess to cover the wire.

13. An endoscope, comprising
a housing;
a wire fixing mechanism according to claim 1, disposed inside the housing; and
a wire, disposed inside the housing, wherein two ends of the wire are respectively fixed to the wire fixing mechanism; two ends of the wire are respectively passed through the fixing members of the base; the securing members are inserted into the fixing members to press again and fix the wire.

14. The wire fixing mechanism according to claim 6, wherein a second glue is filled into the accommodation recess to cover the wire.
